(19) United States
(12) Patent Application Publication
(10) Pub. No.: US 2010/0268091 A1
(43) Pub. Date: Oct. 21, 2010

(54) FLUORESCENCE IMAGING APPARATUS AND ENDOSCOPE APPARATUS

(75) Inventor: Hideyuki Takaoka, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/782,000

(22) Filed: May 18, 2010

Related U.S. Application Data
(63) Continuation of application No. PCT/JP2008/070846, filed on Nov. 17, 2008.

(30) Foreign Application Priority Data
Nov. 30, 2007 (JP) 2007-310984

(51) Int. Cl. A61B 1/06; G01J 1/58; A61B 6/00

(52) U.S. Cl. 600/478; 250/458.1; 600/178; 250/216

(57) ABSTRACT

A site to be observed, such as a lesion, is easily observed by means of bright fluorescence images without increasing the output of a light source. Provided is a fluorescence imaging apparatus (1) including a wideband excitation portion (7) that radiates wideband excitation light capable of exciting a plurality of fluorescent substances contained in a subject (A); a narrow-band excitation portion (8) that radiates narrow-band excitation light capable of exciting at least one fluorescent substance among the fluorescent substances; an excitation-band switching unit (9) that performs switching between the wideband excitation portion (7) and the narrow-band excitation portion (8); and a fluorescence detector (15) having a detection wavelength band enabling detection of any kind of fluorescence from the subject (A) caused by the wideband excitation light and the narrow-band excitation light.

### TECHNICAL FIELD

The present invention relates to fluorescence imaging apparatuses and endoscope apparatuses.

### BACKGROUND ART

Known fluorescence imaging apparatuses in the related art observe agent fluorescence produced by administering a fluorescent agent to an observation site in a living organism and exciting the fluorescent agent (for example, see Patent Citation 1).

In order to remove noise contained in the observed fluorescence due to the shape of the living organism, the distance between the fluorescence imaging apparatus and the living organism, and so forth, this fluorescence imaging apparatus irradiates the observation site in the living organism with excitation light in an excitation wavelength band capable of exciting the fluorescent agent and an autofluorescent substance, detects all fluorescence components emitted from the observation site and a fluorescence component with a wavelength band in part of the wavelength band of the agent fluorescence, and divides them. In other words, in this fluorescence imaging apparatus, all fluorescence components, including the agent fluorescence and autofluorescence, are used as a reference for removing the noise from the agent fluorescence.

Patent Citation 1:

Publication of Japanese Patent No. 3796635

### Disclosure of Invention

However, in a fluorescence imaging apparatus such as an endoscope, when performing fluorescence imaging of a lesion, such as a tumor, inside the body cavity etc. of a living organism, the doctor must identify the lesion while checking a fluorescence image on the monitor. In other words, when a lesion etc. is to be identified to perform fluorescence imaging, although noise must be removed from the agent fluorescence image as in Patent Citation 1, it is first necessary to identify the site to be observed, such as a lesion, at a stage prior to that.

For example, the usual way to identify a lesion etc. using fluorescence from a plurality of fluorescent substances, such as fluorescent agents and autofluorescent substances, involves radiating excitation light having a wavelength capable of exciting a comparatively wide area of a desired fluorescent substance, with the distal end of the fluorescence imaging apparatus away from the surface of the observation site, so as to roughly identify the site where the lesion exists, followed by bringing the distal end of the fluorescence imaging apparatus closer to the roughly identified site to perform more detailed fluorescence imaging.

However, when the distal end is away from the surface of the observation site, because the intensity of the excitation light decreases in inverse proportion to the square of the distance, the fluorescence intensity, which is already weak to begin with, becomes even weaker, causing the fluorescence image to darken and making it difficult to discern the lesion etc. One way that has been considered is to increase the output of the light source to increase the intensity of the excitation light; however, the power consumption is greatly increased, and there are limits to the intensity of excitation light that can be emitted, depending on the capacity of the light source.

Another solution that has been considered is to adjust the fluorescence imaging brightness by changing the characteristics of the filter etc. for fluorescence detection; however, when an image-acquisition unit is provided at the distal end of an observation apparatus, as in an electronic endoscope, because the fluorescence detection filter etc. is also disposed at the distal end of the observation apparatus, it is difficult to change the filter characteristics during observation.

The present invention has been conceived in light of the circumstances described above, and an object thereof is to provide a fluorescence imaging apparatus and endoscope apparatus that are capable of easily performing observation under bright excitation light in the case where a lesion etc. is to be identified while observing a wide area of a subject, and also in the case where the distal end of the fluorescence imaging apparatus is brought close to the subject to perform detailed observation.

In order to realize the object described above, the present invention provides the following solutions.

A first aspect of the present invention is a fluorescence imaging apparatus including a wideband excitation portion that radiates wideband excitation light capable of exciting a plurality of fluorescent substances contained in a subject; a narrow-band excitation portion that radiates narrow-band excitation light capable of exciting at least one fluorescent substance among the fluorescent substances; an excitation-band switching unit that performs switching between the wideband excitation portion and the narrow-band excitation portion; and a fluorescence detector having a detection wavelength band enabling detection of any kind of fluorescence from the subject caused by the wideband excitation light and the narrow-band excitation light.

According to the aspect described above, when the subject is irradiated with wideband excitation light by operating the wideband excitation portion, a plurality of fluorescent substances contained in the subject are simultaneously excited. In cases where a plurality of fluorescent agents that specifically accumulate in a lesion are administered, or in cases where a plurality of fluorescent substances are to be observed, for instance, with a combination of such fluorescent agents and autofluorescent substances that are present in biological tissue, by simultaneously exciting the respective fluorescent substances, it is possible to acquire a fluorescence image in which the lesion is bright, which makes it possible to easily identify a candidate site which is suspected of being a lesion using this fluorescence image. In this state, bringing the identified candidate site to close-up, by switching from the wideband excitation portion to the narrow-band excitation portion by operating the narrow-band excitation portion of the excitation-band switching unit, irradiate the candidate site with narrow-band excitation light capable of exciting a specific fluorescent substance, it is possible to perform even more detailed observation of the lesion.

According to the aspect described above, by simultaneously exciting the plurality of fluorescent substances, it is possible to acquire a fluorescence image in which the candidate site of the lesion shines brightly even when acquiring a wide area of the subject with the fluorescence imaging apparatus away from the subject. Once the candidate site of the lesion is identified, the fluorescence imaging apparatus is brought close to the subject to irradiate a narrow area with the narrow-band excitation light, causing only the specific fluorescent substance that is excited by this narrow-band excitation light to shine brightly, which makes it possible to perform more detailed fluorescence imaging. As a result, it is possible to easily observe a site to be examined, such as a lesion, using a bright fluorescence image, without increasing the output of the light source.

A second aspect of the present invention is a fluorescence imaging apparatus in which at least a portion thereof is inserted inside a body cavity of a living organism to observe fluorescence from a subject inside the body cavity, including a wideband excitation portion that radiates wideband excitation light capable of exciting a plurality of fluorescent substances contained in the subject; a narrow-band excitation portion that radiates narrow-band excitation light that is capable of exciting at least one fluorescent substance of the fluorescent substances and that is contained in an excitation band of the wideband excitation portion; an excitation-band switching unit that performs switching between the wideband excitation portion and the narrow-band excitation portion; an image-acquisition unit that is disposed at a site inserted inside the body cavity and that acquires a fluorescence image from the fluorescent substances; and a fluorescence detector that is disposed between the subject and the image-acquisition unit and that detects fluorescence from the subject caused by the wideband excitation light and the narrow-band excitation light, in a common detection wavelength band.

According to the aspect described above, even with a fluorescence imaging apparatus in which a portion of the observation apparatus is inserted inside the body cavity and the image-acquisition unit is disposed at the tip thereof, as in an electronic endoscope, it is possible to detect fluorescence in a detection wavelength band common to the wideband excitation portion and the narrow-band excitation portion without changing the characteristics of the fluorescence detector. Therefore, in the case of a wide area observation of the subject, and also close observation to the subject, it is possible to easily perform bright fluorescence imaging.

In the aspect described above, a plurality of the narrow-band excitation portions may be provided so as to radiate excitation light in at least two different wavelength bands; and the excitation-band switching unit may also perform switching among the plurality of narrow-band excitation portions.

By doing so, it is possible to perform detailed fluorescence imaging of a candidate site of a lesion with excitation light in two or more different narrow wavelength bands. Also, it is possible to compare the two of more fluorescence images acquired by radiating these excitation light.

The aspect described above may further include an image processing section that controls the switching of the excitation portion by the excitation-band switching unit, and that processes images of fluorescence that is emitted from the subject due to the excitation light radiated from the respective excitation portions and that is acquired by the fluorescence detector, in a manner enabling comparison thereof.

By doing so, by automatically acquiring different fluorescence images in which excitation light is radiated by different excitation portions by controlling the switching of the excitation portions with the excitation-band switching unit, and by performing processing enabling comparison in the image processing section, it is possible to easily observe the subject.

The fluorescence imaging apparatus according to the aspects described above may be employed in an internal observation apparatus in which an objective lens for observing the inside of a living organism is directly inserted therein, for example, an endoscope apparatus, or a microscope apparatus such as that disclosed in Japanese Unexamined Patent Application, Publication No. 2005-241671. By doing so, it is possible to reduce the size of the endoscope apparatus or microscope apparatus, which reduces the pain inflicted in a living organism.

According to the aspects described above, an advantage is afforded in that it is possible to easily perform observation under bright excitation light in the case where a lesion etc. is to be identified while observing a wide area of the subject, and also in the case where the distal end of the fluorescence imaging apparatus is brought close to the subject to perform detailed observation.

### BRIEF DESCRIPTION OF DRAWINGS

FIG. 1 is an overall configuration diagram showing a fluorescence imaging apparatus according to a first embodiment of the present invention.

FIG. 2 is a graph showing wavelength characteristics of individual filters in the fluorescence imaging apparatus in FIG. 1.

FIG. 3 is an overall configuration diagram showing a fluorescence imaging apparatus according to a second embodiment of the present invention.

FIG. 4 is a graph showing wavelength characteristics of individual filters in the fluorescence imaging apparatus in FIG. 3.

FIG. 5 is an overall configuration diagram showing a modification of the fluorescence imaging apparatus in FIG. 3.

FIG. 6 is a graph showing wavelength characteristics of a laser light source and fluorescence filter in FIG. 5.

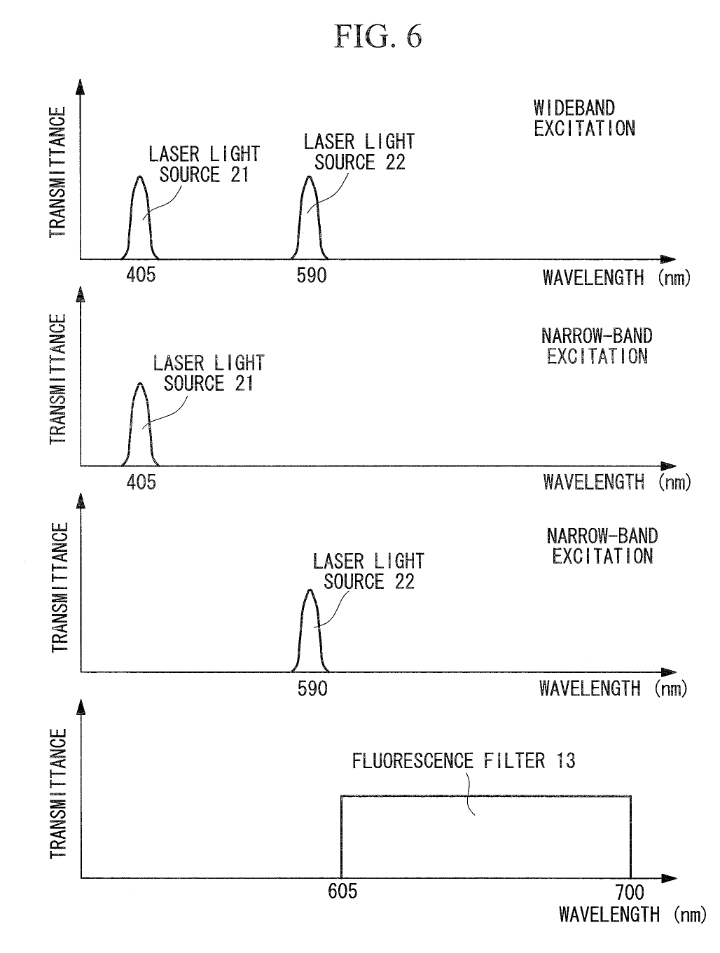

Explanation of Reference:

A: body cavity surface (subject)
1, 20: fluorescence imaging apparatus
4: image processing section
7: wideband excitation filter (wideband excitation portion)
8, 8a, 8b: narrow-band excitation filter (narrow-band excitation portion)
9, 26: excitation-band switching unit
15: image acquisition device (fluorescence detector)

### BEST MODE FOR CARRYING OUT THE INVENTION

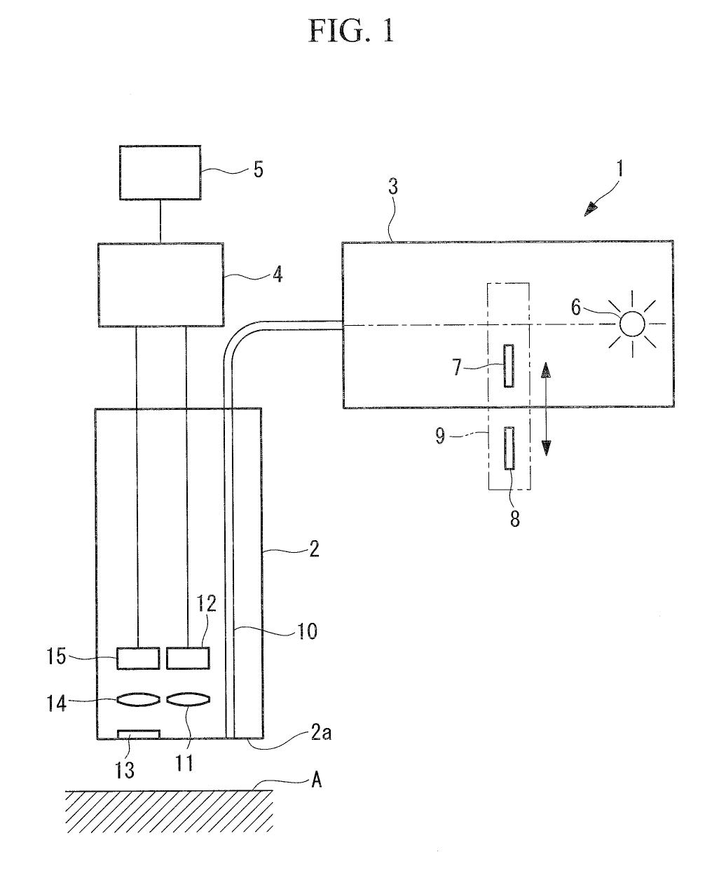

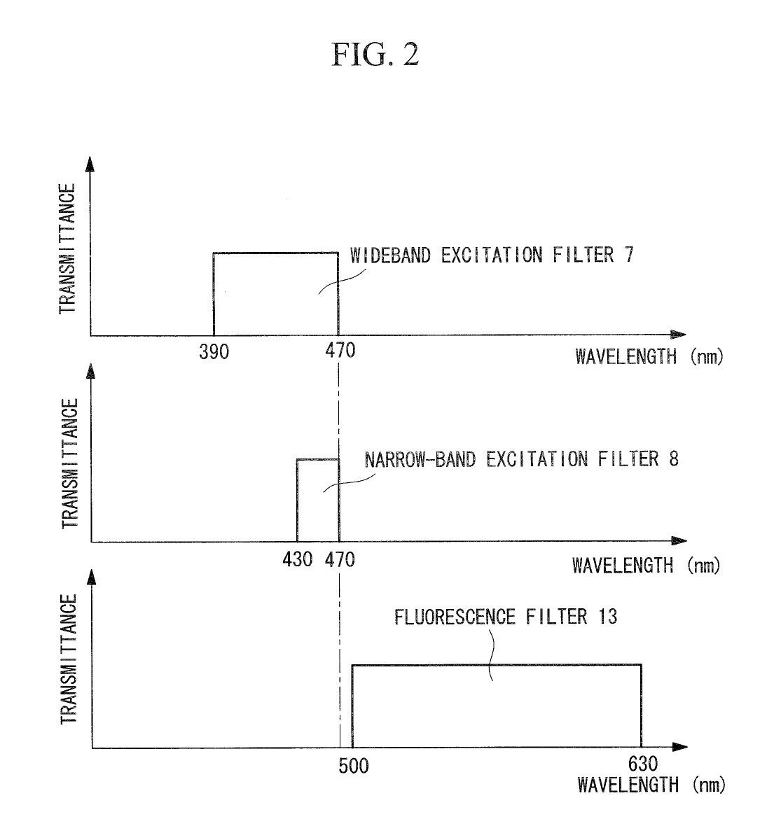

A fluorescence apparatus 1 according to a first embodiment of the present invention will be described below with reference to FIG. 1 and FIG. 2.

As shown in FIG. 1, the fluorescence imaging apparatus 1 according to this embodiment, which is an endoscope apparatus, includes a long, narrow inserted portion 2 that is inserted inside the body cavity of a patient, a light-source apparatus 3 and an image processing apparatus 4 that are connected to the inserted portion 2 and disposed outside the patient's body, and a display device 5 that is connected to the image processing apparatus 4 and that displays an image-processed image.

The light source apparatus 3 includes a white-light source 6 such as a xenon lamp, a wideband excitation filter 7 that transmits excitation light in a wavelength band of 370 to 470 nm among the white light emitted from the white-light source 6, a narrow-band excitation filter 8 that transmits excitation light in a wavelength band of 430 to 470 nm, and an excitation-band switching unit 9 that retracts these excitation filters 7 and 8 from the light path from the white-light source 6 to the inserted portion 2 or alternately disposes them therein.

The inserted portion 2 includes a light guide 10 that guides light from the light-source apparatus 3 to a distal end 2a to emit the light from the distal end 2a. The distal end portion of the inserted portion 2 is provided with a first objective lens 11 that collects reflected light from a body cavity surface A, a reflected-light image acquisition device 12 that acquires the reflected light collected by the first objective lens 11, a fluorescence filter 13 that transmits fluorescence emitted from the body cavity surface A and that blocks excitation light, a second objective lens 14 that collects the fluorescence transmitted through the fluorescence filter 13, and a fluorescence image acquisition device 15 that acquires an image of the fluorescence collected by the second objective lens 14.

As shown in FIG. 2, the fluorescence filter 13 has a transmission wavelength band from 500 to 630 nm.

The image processing apparatus 4 is connected to the two image acquisition devices 12 and 15 disposed at the distal end portion of the inserted portion 2, performs image processing on the reflected-light image and the fluorescence image acquired by these image acquisition devices 12 and 15, and outputs them to the display device 5.

The display device 5 displays the image-processed reflected-light image and fluorescence image either one after another or side-by-side.

The operation of the thus-configured fluorescence imaging apparatus 1 according to this embodiment will be described below.

To perform fluorescence imaging of the patient's body cavity surface A, which is the subject, using the fluorescence imaging apparatus 1 according to this embodiment, first, the operator inserts the inserted portion 2 inside the body cavity of the patient. Then, the operator activates the excitation-band switching unit 9 to retract the narrow-band excitation filter 8 and the wideband excitation filter 7 from the light path from the white-light source 6 to the inserted portion 2 and operates the reflected-light image acquisition device 12.

The white light emitted from the white-light source 6 enters the light guide 10, is guided inside the inserted portion 2 by the light guide 10, and emerges from the distal end 2a of the inserted portion 2. When the emerging white light is radiated onto the body cavity surface A, the light reflected at the body cavity surface A is collected by the first objective lens 11 and is acquired by the reflected-light image acquisition device 12.

The reflected-light image acquired by the reflected-light image acquisition device 12 is subjected to image processing in the image-processing apparatus 4, and is then displayed on the display device 5. Accordingly, it is possible to observe undulations, discoloration, etc. of the body cavity surface A.

Next, the operator activates the excitation-band switching unit 9 to dispose, first of all, the wideband excitation filter 7 in the light path between the white-light source 6 and the inserted portion 2. Then the operator activates the fluorescence image acquisition device 15.

The wideband excitation filter 7 transmits excitation light in a wavelength band of 390 to 470 nm from among the white light emitted from the white-light source 6, and therefore, the excitation light transmitted through the wideband excitation filter 7 is radiated towards the body cavity surface A from the distal end 2a of the inserted portion 2 via the light guide 10. At this time, the distal end 2a of the inserted portion is disposed at a position sufficiently distant from the body cavity surface A so as to radiate the excitation light over a wide area.

Because the radiated excitation light has a wide wavelength band from 390 to 470 nm, it excites various fluorescent substances present in the body cavity surface A, such as substances exhibiting autofluorescence, a fluorescent agent that is administered, etc.

Possible examples of the fluorescent substances that are excited include collagen, elastin, NADH (NADH: reduced nicotinamide adenine dinucleotide), FAD (FAD: flavin adenine dinucleotide), and protoporphyrin. All of these are excited by excitation light in a wavelength band from 390 to 470 nm and emit fluorescence in a wavelength band from 500 to 630 nm.

In other words, when the body cavity surface A is irradiated with a wide excitation light beam with the distal end 2a of the inserted portion 2 away from the body cavity surface A, because the intensity of excitation light reaching the body cavity surface A decreases in inverse proportion to the square of the distance, the individual fluorescent substances can emit only weak fluorescence. However, by exciting various fluorescent substances, it is possible to make various sites on the body cavity surface A shine relatively brightly.

Because the fluorescence filter 13 has a wide transmission wavelength band from 500 to 630 nm, a large amount of the fluorescence produced is transmitted therethrough and is collected by the second objective lens 14, and the excitation light is blocked. Thus, it is possible to acquire a fluorescence image in which candidate sites of lesions shine brightly.

The operator brings the distal end 2a of the inserted portion 2 close to an identified candidate site of a lesion by checking the fluorescence image which covers a comparatively wide area and activates the excitation-band switching unit 9 to dispose the narrow-band excitation filter 8 in the light path between the white-light source 6 and the inserted portion 2. The operating state of the fluorescence image acquisition device 15 is maintained.

Because the narrow-band excitation filter 8 has a transmission wavelength band from 430 to 470 nm, the excitation light in this narrow wavelength band is radiated onto the body cavity surface A, and only a specific fluorescent substance is excited. A possible example of the excited fluorescent substance is FAD. FAD is excited by excitation light in the wavelength band from 430 to 470 nm and emits fluorescence in the wavelength band from 510 to 560 nm.

Therefore, by bringing the distal end 2a of the inserted portion 2 close to the body cavity surface A, only FAD in a comparatively narrow area of the body cavity surface A is excited, and the fluorescence therefrom can be detected.

In this case, because the distal end 2a of the inserted portion 2 is brought close to the body cavity surface A, it is possible to increase the intensity of the excitation light reaching the body cavity surface, even for excitation light in a narrow wavelength band.

Then, for a candidate site of a lesion identified by radiating wideband excitation light, it is possible to perform detailed observation based on only the fluorescence from FAD, which specifically accumulates in tumors etc., and accordingly, it is possible to identify a lesion.

In other words, according to this embodiment, in order to acquire a bright fluorescence image from the FAD, by using an image of a comparatively narrow area with the inserted portion 2 close to the body cavity surface A rather than thoroughly observing the entirety of a wide area on the body cavity surface A, first, a lesion is roughly identified in a fluorescence image in which various kinds of fluorescent substances are excited in a comparatively wide area; therefore, it suffices to perform detailed observation of only the candidate site, which affords an advantage in that the time required for observation can be substantially reduced.

According to this embodiment, the same fluorescence filter may be used when observing with wideband excitation light and when observing with narrow-band excitation light, and therefore, the structure of the distal end portion of the inserted portion 2, where there is not much space, can be simplified, thus allowing a reduction in diameter of the inserted portion 2. A mechanism for changing the fluorescence filter 13 becomes unnecessary, which can also simplify the structure. During observation, an advantage is afforded in that observation can be performed efficiently without having to change the fluorescence filter 13 at the distal end 2a of the inserted portion 2.

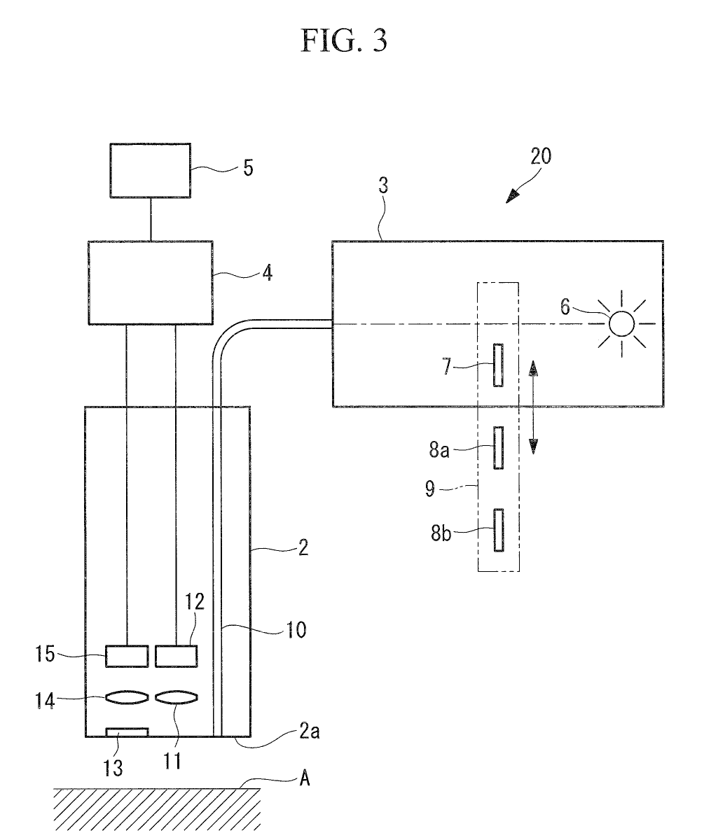

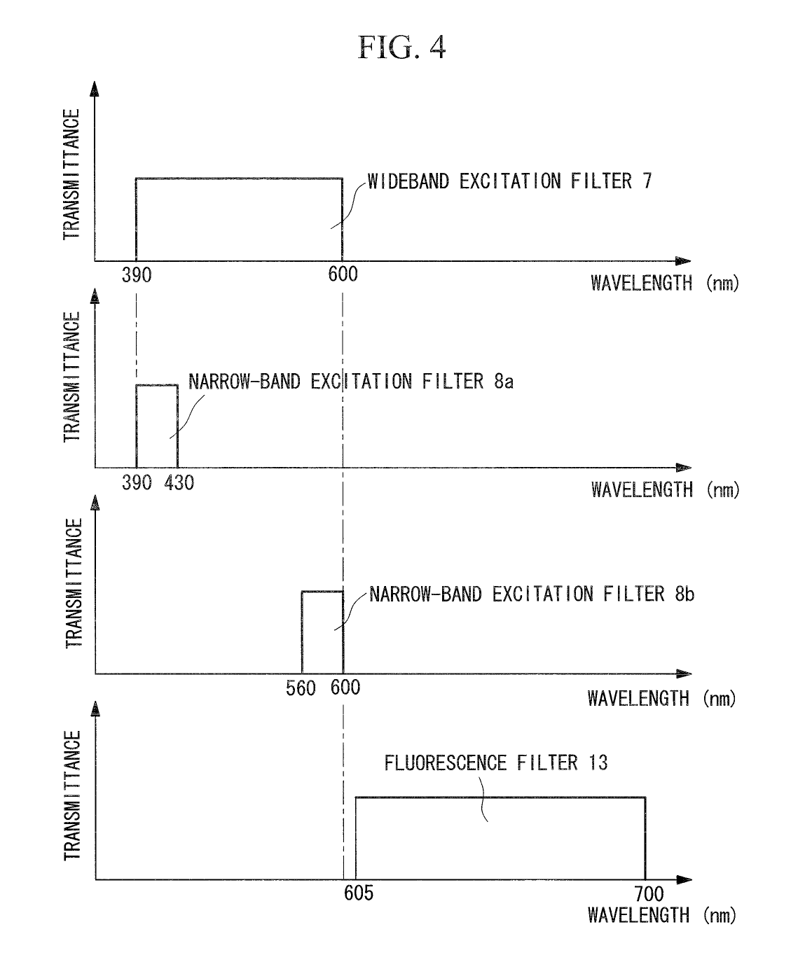

Next, a fluorescence imaging apparatus 20 according to a second embodiment of the present invention will be described with reference to FIGS. 3 and 4.

In the description of this embodiment, parts having the same construction as those in the fluorescence imaging apparatus 1 according to the first embodiment described above are assigned the same reference numerals, and a description thereof will be omitted.

As shown in FIG. 3, the fluorescence imaging apparatus 20 according to this embodiment differs from the fluorescence imaging apparatus 1 according to the first embodiment in that two narrow-band excitation filters 8a and 8b are provided.

The fluorescence imaging apparatus 20 according to this embodiment includes a wideband excitation filter 7 having a wavelength band of 390 to 600 nm, a narrow-band excitation filter 8a having a wavelength band of 390 to 430 nm, and a narrow-band excitation filter 8b having a wavelength band of 560 to 600 nm. The transmission wavelength band of the fluorescence filter 13 is from 605 to 700 nm.

To perform fluorescence imaging using the fluorescence imaging apparatus 20 according to this embodiment, two fluorescent agents are injected into the tissue of the body cavity surface A serving as the subject. As the fluorescent agent, a fluorescent probe in which ALA (ALA: aminolevulinic acid), which is a substance exhibiting affinity to tumors, is composed and a fluorescent probe in which Texas Red (trade name) is partially composed are used. The latter fluorescent probe is endowed with properties whereby it binds to biomolecules found particularly in lesions, such as tumors etc.

Protoporphyrin IX is derived from ALA. Protoporphyrin IX can be excited most efficiently around 405 nm and emits fluorescence in the wavelength band 620 to 700 nm.

Texas Red can be excited most efficiently around 590 nm and emits fluorescence in the wavelength band 600 to 650 nm.

With the thus-configured fluorescence imaging apparatus 20 according to this embodiment, like the fluorescence imaging apparatus 1 according to the first embodiment, the position of the distal end 2a of the inserted portion 2 is checked with white light, and then, using the wideband excitation filter 7, a candidate site is identified with a fluorescence image in which the candidate sites of lesions covering a wide area shine brightly. Then, at the identified candidate site, it is possible to observe a specific fluorescent substance that is excited by each excitation wavelength by switching between the two narrow-band excitation filters 8a and 8b.

In other words, according to this embodiment, in addition to the advantages of the fluorescence imaging apparatus 1 according to the first embodiment, it is possible to perform calculations between the fluorescence images acquired while switching between the wideband excitation filter 7 and the two narrow-band excitation filters 8a and 8b, and to display the image resulting from those calculations. For example, by calculating the ratio of the fluorescence images acquired while switching between the two narrow-band excitation filters 8a and 8b, in the observation region, it is possible to show, in a manner allowing comparison, which of the two kinds of fluorescent agent exhibits a large reaction. A plurality of fluorescence images may be displayed side-by-side.

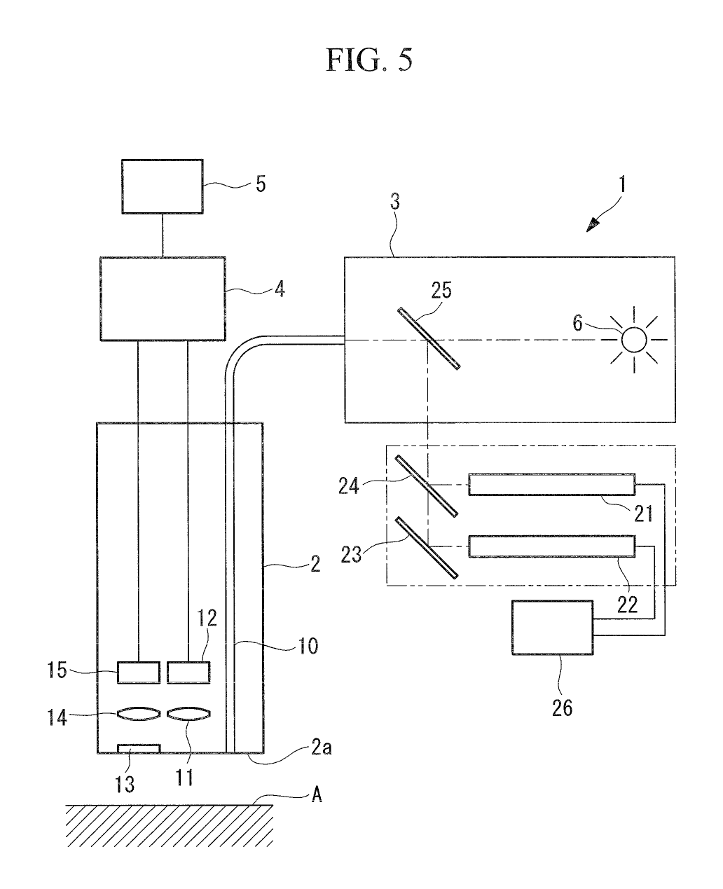

In the fluorescence imaging apparatuses 1 and 20 according to each of the embodiments described above, the excitation-band switching unit 9 switches the wavelength of the excitation light by replacing the excitation filters 7, 8, 8a, and 8b. Instead of this, however, as shown in FIG. 5, it is possible to employ a light-source apparatus 3 in which a plurality of excitation light sources emitting excitation light of different wavelengths, for example, laser light sources 21 and 22, are combined. Reference numeral 23 in the figure is a mirror, reference numeral 24 is a dichroic mirror, and reference numeral 25 is a half-mirror.

For example, laser light with a center wavelength of 405 nm should be emitted from the first laser light source 21, laser light with a center wavelength of 590 nm should be emitted from the second laser light source 22, and both types of laser light should be simultaneously emitted in the case of observation where wideband excitation light is radiated.

In this case, with an excitation-band switching unit 26, each of the laser light sources 21 and 22 may be switched on and off, or shutters (not shown) disposed in front of the emission openings of the respective laser light sources 21 and 22 may be switched on and off.

By employing a control device (not shown) that performs, at a prescribed timing, either switching between the wideband excitation filter 7 and the narrow-band excitation filters 8, 8a, and 8b or switching among the narrow-band excitation filters 8a and 8b, image processing that enables comparison based on the ratio or difference between the fluorescence images acquired while switching the wavelength of the excitation light may be performed automatically.

1. A fluorescence imaging apparatus comprising:
a wideband excitation portion that radiates wideband excitation light capable of exciting a plurality of fluorescent substances contained in a subject;
a narrow-band excitation portion that radiates narrow-band excitation light capable of exciting at least one fluorescent substance among the fluorescent substances;
an excitation-band switching unit that performs switching between the wideband excitation portion and the narrow-band excitation portion; and
a fluorescence detector having a detection wavelength band enabling detection of any kind of fluorescence from the subject caused by the wideband excitation light and the narrow-band excitation light.

2. A fluorescence imaging apparatus in which at least a portion thereof is inserted inside a body cavity of a living organism to observe fluorescence from a subject inside the body cavity, comprising:
a wideband excitation portion that radiates wideband excitation light capable of exciting a plurality of fluorescent substances contained in the subject;
a narrow-band excitation portion that radiates narrow-band excitation light that is capable of exciting at least one fluorescent substance of the fluorescent substances and that is contained in an excitation band of the wideband excitation portion;
an excitation-band switching unit that performs switching between the wideband excitation portion and the narrow-band excitation portion;
an image-acquisition unit that is disposed at a site inserted inside the body cavity and that acquires a fluorescence image from the fluorescent substances; and
a fluorescence detector that is disposed between the subject and the image-acquisition unit and that detects fluorescence from the subject caused by the wideband excitation light and the narrow-band excitation light, in a common detection wavelength band.

3. A fluorescence imaging apparatus according to claim 1, wherein
a plurality of the narrow-band excitation portions are provided so as to radiate excitation light in at least two different wavelength bands; and
the excitation-band switching unit also performs switching among the plurality of narrow-band excitation portions.

4. A fluorescence imaging apparatus according to claim 1, further comprising an image processing section that
controls the switching of the excitation portion by the excitation-band switching unit, and
processes images of fluorescence that is emitted from the subject due to the excitation light radiated from the respective excitation portions and that is acquired by the fluorescence detector, in a manner enabling comparison thereof.

5. An endoscope apparatus comprising the fluorescence imaging apparatus according to claim 1.
